(19) United States
(12) Patent Application Publication
(10) Pub. No.: US 2002/0034724 A1
(43) Pub. Date: Mar. 21, 2002

(54) Methods to selectively inactivate microorganisms in biological compositions

(76) Inventors: Clark M. Edson, Watertown, MA (US); Andrei A. Purmal, Waltham, MA (US); Samuel K. Ackerman, Weston, MA (US)

Correspondence Address: CLARK & ELBING LLP, 176 FEDERAL STREET, BOSTON, MA, 02110-2214, US

(21) Appl. No.: 09/877,838

(22) Filed: Jun. 8, 2001

Related U.S. Application Data
(63) Continuation of application No. 09/161,030, filed on Sep. 25, 1998.
Application No. 09/161,030 is a continuation-in-part of application No. 08/855,378, filed on May 13, 1997, now Pat. No. 6,136,586.
Application No. 08/855,378 is a continuation-in-part of application No. 08/705,045, filed on Aug. 29, 1996, now abandoned.
Application No. 08/705,045 is a continuation-in-part of application No. 08/521,245, filed on Aug. 29, 1995, now Pat. No. 6,114,108.

(51) Int. Cl. C12N 5/08

(52) U.S. Cl. 435/2; 435/372

(57) ABSTRACT

The invention features a method for inactivating microorganisms such as viruses and bacteria in a preparation of purified mammalian cells which, when mature, lack a nucleus (e.g., red blood cells or platelets). The method includes contacting the preparation with ethyleneimine dimer under viral inactivating conditions for a period of time sufficient to inactivate at least some of the microorganismsin the preparation. Also disclosed is a method for removing ethyleneimine dimer from a treated biological composition following inactivation, without the addition of any quenching agent.

### CROSS-REFERENCE TO RELATED APPLICATIONS

This application is a continuation of U.S. Ser. No. 09/161,030, filed Sep. 25, 1998 (now pending), which is a continuation-in-part of U.S. Ser. No. 08/855,378 (now U.S. Pat. No. 6,136,586), which is a continuation-in-part of U.S. Ser. No. 08/705,045 (now abandoned) which is a continuation-in-part of U.S. Ser. No. 08/521,245 (now U.S. Pat. No. 6,114,108), each of which is hereby incorporated by reference.

### BACKGROUND OF THE INVENTION

This invention relates to methods and compositions for the selective inactivation of viruses, bacteria, and other microorganisms in biological compositions, such as blood.

Following traumatic injury (or during surgery), an organism may require a blood transfusion to prevent death due to blood loss. In humans and certain domesticated animals, blood transfusion has enabled the survival of injured individuals who would otherwise have died from blood loss.

Whole blood is composed of many different types of proteins and cells. Blood proteins include antibodies, complement proteins, and proteins involved in the blood clotting cascade. In addition, each of the different types of blood cells plays a unique role in maintaining the health of the organism. Red blood cells, for instance, are essential for the transport of oxygen and carbon dioxide gases to and from the cells of a multicellular organism. Another type of blood cell, a platelet, is involved in initiating blood clotting; thrombocytopenia patients have a platelet deficiency and are prone to bleeding disorders.

One caveat in using blood transfusions is the danger of transmitting blood-borne viruses from donor blood to a recipient. The transmission of viral diseases (e.g., hepatitis A, B, and C, acquired immunodeficiency syndrome, and cytomegalovirus infections) by blood or blood products is a significant problem in medicine. Screening donor blood for viral markers can help reduce the transmission of viruses to recipients, but many screening methods are directed to only a few discrete viruses and are therefore incomplete or less than 100% sensitive.

A number of agents have been developed that are capable of inactivating viruses found in blood, as well as viruses found in other biological compositions, such as mammalian and hybridoma cell lines, products of cell lines, milk, colostrum, and urine. For example, ethyleneimine monomer and ethyleneimine oligomers are very effective viral inactivating agents. These agents are themselves toxic, and must therefore be rendered non-toxic before a product, such as blood or milk proteins, may be used clinically. Typically, a viral inactivating compound, such as ethyleneimine dimer, is added to a biological composition to inactivate infectious viruses that might be present in the composition. A quenching agent is then added to inactivate the ethyleneimine dimer that remains after viral inactivation has taken place. The end result is a biological composition that is relatively free of infectious viruses, but that is contaminated with quenched inactivating agent and with quenching agent.

### SUMMARY OF THE INVENTION

In general, the present invention features a method for inactivating viruses, bacteria, or other microorganisms in purified mammalian enucleated cell preparations, and a method for removing an inactivating agent from a treated biological composition by washing with an inert solution.

Accordingly, in a first aspect, the invention features a method of inactivating viruses or other microorganisms in a preparation of purified mammalian enucleated cells that includes contacting the preparation with ethyleneimine dimer under viral inactivating conditions for a period of time sufficient to inactivate at least some of the microorganisms in the preparation.

In various embodiments, the microorganisms are viruses and at least 90% of the viruses in the preparation are inactivated, or preferably at least 98% of the viruses in the preparation are inactivated. In another embodiment, the cell is a human cell.

In another embodiment of the first aspect, the method further includes washing the contacted preparation with a solution that does not quench the ethyleneimine dimer, where the washing reduces the concentration of the ethyleneimine dimer in the washed preparation. Preferably, the concentration of the ethyleneimine dimer in the washed preparation is at or below a concentration of the ethyleneimine dimer that is toxic. In other embodiment, the solution is sterile unbuffered saline and the washing is manual.

In another embodiment of the first aspect, the washing includes includes the steps of: (i) layering the contacted preparation on a mesh having pores with diameters smaller than the diameters of the enucleated cells; and (ii) continuously flowing the solution that does not quench the ethyleneimine dimer over the contacted preparation. In yet another embodiment, the washing includes the steps of: (i) adding a volume of the solution that is at least three times the volume of the preparation; and (ii) removing the solution from the preparation. Preferably, the washing is repeated at least two times.

In another embodiment of the first aspect, the washing is automated. Preferably, in the washing process, a container containing the contacted preparation is in a machine that performs the following steps under sterile conditions: (i) pumping the preparation out of the container; (ii) diluting the preparation with the solution that does not quench the ethyleneimine dimer; (iii) removing the solution from the preparation, wherein the solution is discarded; and (iv) pumping the preparation back into the container. Preferably, the machine performs steps (ii) and (iii) at least two times.

In another embodiment of the first aspect of the invention, the method further includes quenching the contacted preparation with a quenching agent. The quenching agent may be soluble or may be immobilized on a solid-phase support.

In a second aspect, the invention features a method for selectively inactivating animal viruses in a biological composition that includes the steps of: (a) contacting the composition with an ethyleneimine dimer under viral inactivating conditions for a period of time sufficient to inactivate at least some of the animal viruses in the composition; and (b) washing the composition with a solution that does not quench the ethyleneimine dimer, wherein the washing reduces the amount of the ethyleneimine dimer in the composition. Preferably, the concentration of the ethyleneimine dimer in the washed composition is at or below a concentration of the ethyleneimine dimer that is toxic. In various embodiments, the solution is sterile unbuffered saline and the washing step is automated.

In yet another embodiment of the second aspect of the invention, in the washing step, a container containing the contacted composition is in a machine that performs the following steps under sterile conditions: (i) pumping the composition out of the container; (ii) diluting the composition with the solution that does not quench the ethyleneimine dimer; (iii) removing the solution from the composition, wherein the solution is discarded; and (iv) pumping the composition back into the container. Preferably, the machine performs steps (ii) and (iii) at least two times.

In yet another embodiment of the second aspect, the washing step includes: (i) adding a volume of the solution that is at least three times the volume of the composition; and (ii) removing the solution from the composition. Preferably, the washing step is repeated at least two times.

In various other embodiments of the second aspect, the composition includes a mammalian cell and the washing step includes: (i) layering the contacted composition on a mesh having pores with diameters smaller than the diameter of the mammalian cell; and (ii) continuously flowing the solution that does not quench the ethyleneimine dimer over the contacted composition. The mammalian cell may be an enucleated cell (e.g., a platelet or a red blood cell). In other embodiments, the composition is a cell-free composition and the washing step is manual.

From the description of the invention, it will be realized that the ethyleneimine dimer inactivating agents and methods of this invention can be used to inactivate blood-transmitted viruses, bacteria, or parasites in cell- or biopolymer-containing compositions in various contexts, e.g., in the hospital, laboratory, as part of a kit. Since cell compositions also comprise a variety of proteins, the method of viral inactivation described herein is also applicable to protein fractions, particularly blood plasma protein fractions or purified blood products, including, but not limited to, fractions containing clotting factors (such as factor VIII and factor IX), serum albumin and/or immunoglobulins. The viral and bacterial inactivation may be accomplished by treating a protein fraction or purified protein with ethyleneimine dimer as described herein.

By “purified” is meant a preparation that contains, by volume, at least 50%, more preferably, at least 70%, more preferably at least 85%, even more preferably at least 95%, and most preferably, at least 98% of the indicated component. For example, a purified preparation of red blood cells contains at least 50% by volume red blood cells.

By “ethyleneimine dimer” is meant a compound having the following formula:

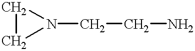

By “nucleic acid” is meant both DNA and RNA, both single and double stranded.

“Inactivating,” “inactivation,” or “inactivate,” when referring to nucleic acids, means to substantially eliminate the template activity of DNA or RNA, for example, by destroying the ability to replicate, transcribe or translate a message. For example, the inhibition of translation of an RNA molecule can be determined by measuring the amount of protein encoded by a definitive amount of RNA produced in a suitable in vitro or in vivo translation system. When referring to viruses, the term means diminishing or eliminating the number of infectious viral particles measured as a decrease in the infectious titer or number of infectious virus particles per ml. Such a decrease in infectious virus particles is determined by assays well known to a person of ordinary skill in the art.

“Viral inactivating conditions” refers to the conditions under which the viral particles are incubated with ethyleneimine dimer, including, for example, time of treatment, pH, temperature, salt composition and concentration of ethyleneimine dimer so as to inactivate the viral genome to the desired extent. Viral inactivating conditions are selected from the conditions for selective modification of nucleic acids described in U.S. Pat. No. 6,136,586, hereby incorporated by reference.

By “inactivate at least some of the animal viruses” is meant that at least 50% of the viruses in the treated preparation are inactivated, preferably at least 70% of the viruses are inactivated, more preferably at least 80%, still more preferably at least 90%, still more preferably at least 95%, still more preferably, at least 99%, and most preferably, 100% of the viruses in the treated preparation are inactivated. The number of viruses in a preparation may be measured by the number or titer of infectious viral particles per ml of preparation. Such a measurement may be accomplished by a variety of well known virus titer assays.

By “animal virus” is meant a virus capable of infecting a cell from an animal. Animal viruses may be DNA or RNA viruses, and may be enveloped or non-enveloped viruses or viroids. Examples of animal viruses include, without limitation, poxviruses, herpes viruses, adenoviruses, papovaviruses, parvoviruses reoviruses, orbiviruses, picomaviruses, rotaviruses, alphaviruses, rubiviruses, influenza viruses, type A and B, flaviviruses, coronaviruses, paramyxoviruses, morbilliviruses, pneumoviruses, rhabdoviruses, lyssaviruses, orthmyxoviruses, bunyaviruses, phleboviruses, nairoviruses, hepadnaviruses, arenaviruses, retroviruses, enteroviruses, rhioviruses and the filoviruses. Specifically excluded from the definition of an animal virus are viruses which infect non-animal cells (e.g., a bacteriophage which infects bacterial cells).

By “biological composition” is meant a composition containing cells or a composition containing one or more biological molecules, or a composition containing both cells and one or more biological molecules. Cell-containing compositions include, for example, mammalian blood, red cell concentrates, platelet concentrates, blood plasma, platelet-rich plasma, placental extracts, mammalian cell culture or culture medium, products of fermentation, and ascites fluid. Biological compositions may also be cell-free, and contain at least one biological molecule. By “biological molecule” is meant any class of organic molecule normally found in living organisms including, for example, nucleic acids, polypeptides, post-translationally modified proteins (e.g., glycoproteins), polysaccharides, and lipids. Biological molecule-containing biological compositions include, for example, serum, blood cell proteins, blood plasma concentrate, blood plasma protein fractions, purified or partially purified blood proteins or other components, a supernatant or a precipitate from any fractionation of the plasma, purified or partially purified blood components (e.g., proteins or lipids), mammalian colostrum, milk, urine, saliva, a cell lysate, cryoprecipitate, cryosupematant, or portion or derivative thereof, compositions containing proteins induced in blood cells, and compositions containing products produced in cell culture by normal or transformed cells (e.g., via recombinant DNA or monoclonal antibody technology).

By an “enucleated cell” is meant a cell which, when mature, lacks a nucleus. Preferred examples of enucleated cells are platelets and red blood cells.

By a “solution that does not quench an ethyleneimine dimer” is meant a solution that does not contain a quenching agent (e.g., a thiophosphate or a thiosulfate). A quenching agent, when contacted with ethyleneimine dimer, renders the contacted ethyleneimine dimer non-toxic. Preferred solutions that are incapable of reacting with an ethyleneimine dimer are unbuffered saline and water.

By a “quenching agent” is meant a thiophosphate or a thiosulfate, or a compound containing a thiophosphate or a thiosulfate that, when contacted with ethyleneimine dimer, is capable of rendering the contacted ethyleneimine dimer non-toxic.

It is an object of this invention to provide methods and compositions which allow selective modification of nucleic acid in the presence of other valuable biological macromolecules and cells. According to the methods and compositions of this invention, the nucleic acid of viruses, other microorganisms and cells, are selectively chemically modified, while preserving structure and function of non-nucleic acid components. It is also an object of the invention to provide selective inactivating agents to inactivate the virus, bacterium, or parasite while preserving their immunogenicity and achieving maximum reproducibility.

Currently, red blood cell preparations are used to transfuse a recipient, without prior inactivation of viruses. Hence, the methods and compositions of the present invention allow the inactivation of viruses in red blood cell preparations prior to their use in transfusions. As the inactivating agents described herein are selective for the nucleic acids that are a major component of viruses, viral nucleic acid can be selectively inactivated over the other molecules (e.g., proteins and lipids) present in the red blood cell preparation.

In addition, the invention features a method for removing ethyleneimine dimer from a biological composition following virus inactivation without using a quenching agent. This method results in a biological composition that is relatively free not only of contaminating viruses, but also relatively free of quenched (i.e., non-toxic) ethyleneimine dimer and unreacted quenching agent.

Other features and advantages of the invention will be apparent from the following description and from the claims.

### BRIEF DESCRIPTION OF THE DRAWINGS

FIG. 1 is a schematic diagram showing the chemical reactions that take place during the post-column derivatization of ethyleneimine dimer or quenched ethyleneimine dimer.

FIG. 2 is a schematic flow diagram showing the post-column reaction hardware used in the HPLC analysis of ethyleneimine oligomers.

FIG. 3 is the HPLC elution profile of ethyleneimine dimer.

FIG. 4 is a bar graph showing the distribution of ethyleneimine dimer between the red blood cell component and the plasma component of fresh baboon whole blood (spun hematocrit (HCT) of 38%) following treatment with 12 mM (1000 μg/ml; 0.1% v/v) ethyleneimine dimer for six hours at 20° C.

FIG. 5 is a bar graph showing the effectiveness of manual washing of ethyleneimine dimer from the red blood cell fraction of whole baboon blood (spun HCT 38%) following treatment with 12 mM ethyleneimine dimer for six hours at 20° C. The number of manual washes of the RBC fraction with nonbuffered saline (1:10 v/v) for 10 min. at 20° C. is indicated, and residual ethyleneimine dimer in the RBC fraction is shown as a percentage.

FIG. 6 is a bar graph showing the effectiveness of manual washing of ethyleneimine dimer from the red blood cell fraction of whole baboon blood (SPUN HCT 38%) following treatment with 1000 μg/ml ethyleneimine dimer for six hours at 20° C. The number of manual washes of the RBC fraction with nonbuffered saline (1:10 v/v) for 10 min. at 20° C. is indicated, and residual ethyleneimine dimer in the RBC fraction is shown as μg of ethyleneimine dimer per ml of RBC.

FIG. 7 is a bar graph showing the quenching of the ethyleneimine dimer (6 mM) from human blood (plasma (gray bar) and red blood cells (black bar)) following incubation for two hours at 23° C. with either 50 mM Na-thiosulfate or an equimolar amount of AgroPore-Thiophosphate solid-phase (a solid-phase quencher containing thiophosphomonoester groups).

### DETAILED DESCRIPTION

We have discovered a method to selectively inactivate microorganisms in enucleated cell preparations by treating the preparations with ethyleneimine dimer. For example, most mature mammalian red blood cells, unlike those of other vertebrate animals, lack nuclei and, hence, lack nucleic acid. Thus, treatment of the cells with ethyleneimine dimer that inactivates nucleic acids allows for the selective inactivation of the nucleic acid of any viruses contaminating the red blood cell preparation, while leaving the red blood cells unaffected. Similarly, if the functional nucleic acid is bacterial or is part of another organism's genome, the methods are useful in disinfecting or eliminating such bacteria or other organism. Accordingly, as we demonstrate below, ethyleneimine dimer-mediated inactivation of nucleic acids in red blood cell preparations does not affect the in vivo longevity of the cells. Likewise, since mature platelet cells (also known as platelets) lack nuclei, they are similarly unaffected by treatment with virus-inactivating ethyleneimine dimer.

The invention also provides a method for removing the ethyleneimine dimer from the treated biological composition (e.g., blood), prior to use of the composition by repeatedly washing the composition with a solution that does not quench the ethyleneimine dimer (e.g., sterile unbuffered saline). Where the biological composition is a composition containing cells, the treated cells may be washed by repeated steps of resuspension in a solution that does not quench an ethyleneimine dimer and isolating the cells by centrifugation. Where the biological composition is a cell-free composition (e.g., milk), the treated milk proteins may be, for example, diluted with a solution that does not quench an ethyleneimine dimer, and then dialyzed to remove the ethyleneimine dimer.

Thus, unlike the current methods which inactivate ethyleneimine dimer in a treated biological composition with a quenching agent, leaving the biological composition contaminated with the quenched ethyleneimine dimer and extraneous quenching agent, the method of the present invention allows the generation of a biological composition free of both virus and quenching agent.

### Enucleated Cell Preparation

Since the goal of a blood transfusion is often the transfer of red blood cells, it may be desirable to separate these cells from the other blood components, such as white blood cells (e.g., lymphocytes, neutrophils, and platelets) and biological molecules (e.g., clotting factors and complement). In one example, prior to transfusion, whole blood may be separated into the following components: (1) the red blood cell (RBC) portion (which includes a small portion of the white blood cells) and (2) the plasma (which also includes a small portion of the white blood cells).

Standard methods exist for the separation of red blood cells from other blood components. For example, a Ficoll or Percoll gradient may be used to separate the different components of whole blood based on their differences in density. Such gradients may be generated using reagents commercially available from, for example, Pharmacia Biotech (Uppsala, Sweden).

In addition, commercially available systems, such as the MCS®+ Apheresis System (commercially available form Haemonetics Corp., Braintree, Mass.) may be used to isolate red blood cells from whole blood. It should be noted that this system may also be used to separate other enucleated cells (e.g., platelet cells) from whole blood.

### Removal of Ethyleneimine Dimer from a Treated Biological Composition

Although ethyleneimine dimer is a useful compound for the selective inactivation of viral nucleic acids, its inherent alkylating abilities may render it toxic to most nucleated cells. Thus, prior to introduction of the treated red blood cells into the recipient animal, it is desirable to remove the ethyleneimine dimer from the cells, or at least reduce the concentration of the ethyleneimine dimer to a level that is non-toxic. Of course, if the cells or biological molecules are to be used in vivo, the washing step, in addition to the ethyleneimine dimer treatment step, must be conducted under sterile conditions.

We have employed a murine lymphoma mutagenesis assay to detect toxic (i.e., mutagenic) levels of ethyleneimine dimer and have found that the toxic level for ethyleneimine dimer is greater than 1 μg/ml (i.e., a concentration of 1 μg/ml ethyleneimine dimer or less is non-toxic). Hence, the goal of the washing step is to reduce the concentration of ethyleneimine dimer in the desired treated biological composition to a level at or below that determined to be non-toxic.

One method to remove ethyleneimine dimer from a treated red blood cell preparation is to subject the cells to repeated washings with nonbuffered sterile saline (i.e., sterile 0.9% NaCl). Following each washing step, a sample of the biological composition being treated (and washed) may be tested for the presence of the ethyleneimine dimer at a concentration higher than that previously determined to be toxic. If the concentration is found to be at a toxic level, at the very minimum, at least one additional washing step should be performed prior to the in vivo use of the treated composition. As a safety measure, once a non-toxic level of ethyleneimine dimer is achieved, an additional washing step is preferably performed prior to the in vivo use of the treated composition.

In one example of washing red blood cells (RBCs) following treatment of whole blood, the treated whole blood is diluted with approximately 3X volume of sterile 0.9% NaCl (i.e., 15 ml saline is added to 5 ml blood). Following centrifugation to isolate the RBC component, the packed RBC volume is resuspended in approximately 9X volume of sterile 0.9% NaCl, and allowed to mix (under gentle mechanical agitation) for 10 minutes at 22° C. The RBC component is then isolated by centrifugation, and the washing step with 9X volume of sterile saline is repeated until the concentration of ethyleneimine dimer in the RBC component is at or below the concentration determined to be non-toxic (as determined, for example, using the mouse lymphoma mutagenicity assay described below).

In a second example, isolated platelets (isolated using, for example, plateletpheresis at a standard blood bank facility), are treated with a concentration of ethyleneimine dimer for an amount of time and at an incubation temperature sufficient to inactivate at least some of the viruses in the platelet preparation. The platelets are next repeatedly washed in at least 4X volume of sterile saline solution until the concentration of ethyleneimine dimer is at or below the concentration determined to be non-toxic.

In another example, where the composition contains cells having a known diameter, the cells may be collected in mesh having pores of a diameter smaller than the cells' diameter, and then washed under a continuous flow of a solution that does not quench ethyleneimine dimer for a period of time sufficient to lower the concentration of ethyleneimine dimer in the cells to a non-toxic level. Of course, the fewer cells layered on the mesh, the lower the period of time necessary to wash the cells under a continuous flow. In a variation of this washing method, the ethyleneimine dimer-treated cells may collected in a mesh bag having pores of a diameter smaller than the cells' diameter. The bag may then be repeatedly dipped in a solution that does not quench the ethyleneimine dimer until the concentration of the ethyleneimine dimer in the cells is reduced to a non-toxic level.

Although the washing steps described in the following examples are manual washings under sterile conditions, it will be understood that automated washing may be employed to free a biological composition from an ethyleneimine dimer. For example, a machine may be designed to wash ethyleneimine dimer treated cells under sterile conditions.

In one example of such a machine, purified platelets may be treated with ethyleneimine dimer under viral inactivating conditions for a period of time sufficient to inactivate at least some of the microorganisms in the platelet preparation. This treatment step may be performed by combining the platelets with the ethyleneimine dimer in a sterile container, such as a sterile plastic bag. The bag may then be attached to the machine such that machine can, under sterile conditions, pump the cells out of the bag (and, additionally rinse the bag with sterile 0.9% NaCl). Under completely sterile conditions, the machine may then dilute the platelets with sterile saline, gently mix the platelets for a desired time at a desired temperature, collect the platelets by centrifugation, discard the “used” sterile 0.9% NaCl, and add “fresh” 0.9% NaCl, and repeat the mixing-centrifugation-discarding process for a desired number of times. After the final collection of the platelets by centrifugation, the platelets may be resuspended in “fresh” 0.9% NaCl, or in another desired solution (e.g., blood), and returned to the original container. Platelets thus virally inactivated and washed may be used immediately, stored, or frozen as desired.

### Toxicity Screening of an Ethyleneimine Dimer

The same alkylating abilities of ethyleneimine dimer that render it able to inactivate viral nucleic acids also enable it to damage and/or induce mutations in the genomic DNA of mammalian cells. Hence, before an ethyleneimine dimer-treated biological composition may be used in vivo, it is desirable to reduce the concentration of ethyleneimine dimer in the composition to a level that is non-toxic to most mammalian cells.

The mouse lymphoma mutagenicity assay is one method to determine the toxicity concentration level of ethyleneimine dimer. This assay uses a murine lymphoma cell which is heterozygous at the thymidine kinase (TK) locus (i.e., TK +/−) grown in the presence of the toxin, 5-trifluorothymidine (TFT), to screen different concentrations of ethyleneimine dimer. Both TK +/− cells and TK −/− cells are viable in normal culture media; however, in the presence of TFT, only the TK −/− cells will grow because the TK +/− cells will incorporate the toxic TFT into their DNA. If the TK+/− murine lymphoma cells are exposed to a toxic concentration of ethyleneimine dimer, they may undergo a single-step forward mutation to a TK−/− genotype, enabling them to grow in the presence of TFT. Thus, a concentration of ethyleneimine dimer which does not result in the growth of TK +/− murine lymphoma cells in the presence of 5-trifluorothymidine (TFT) is non-toxic.

The mutagenicity test we used below to screen the toxicity level of ethyleneimine dimer was performed by Convance Laboratories Inc. (Vienna, Va.). L5178Y murine lymphoma cells (which are heterozygous at the TK locus) grown in the presence of TFT were exposed to different concentrations of ethyleneimine dimer. The results demonstrated that a concentration of 1 μg/ml ethyleneimine dimer did not allow the growth (i.e., the forward mutation) of L5178Y TK +/− cells in the presence of TFT. Hence, a concentration of less than or equal to 1 μg/ml ethyleneimine dimer is non-toxic, and a ethyleneimine dimer-treated biological composition (e.g., a treated RBC preparation) containing up to 1 μg/ml ethyleneimine dimer is safe for infusion into a recipient.

### High Performance Liquid Chromatography (HPLC) Analysis of Ethyleneimine Dimer

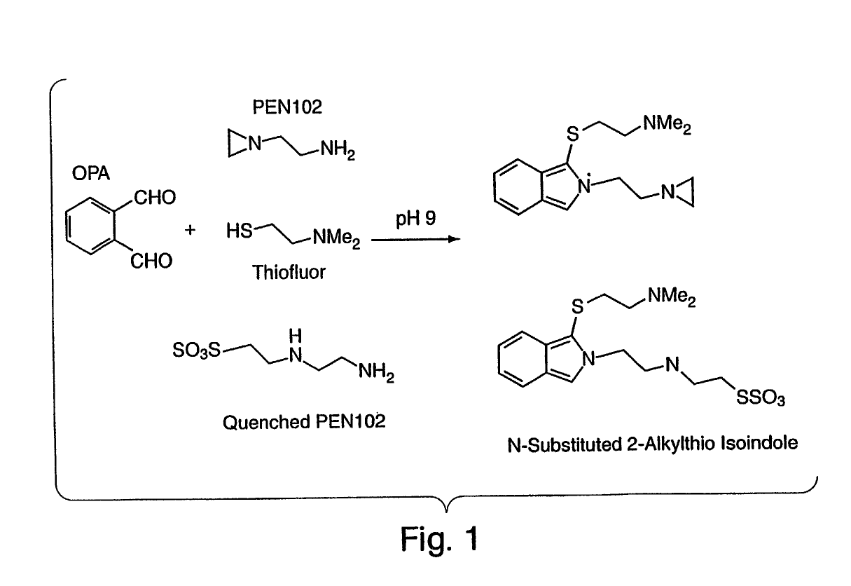

Ethyleneimine oligomers are routinely analyzed by cation exchange HPLC. Because ethyleneimine oligomers do not contain a chromophore, the following method uses post-column derivatization which reacts only with primary amino groups, thereby eliminating complications of multiple reaction sites (ethyleneimine oligomers contain only one primary amino group) or interfering compounds. A schematic of the reactions that take place during HPLC of ethyleneimine dimer and quenched ethyleneimine dimer is shown on FIG. 1. The method has been determined to be linear within the range of 10-230 ng of ethyleneimine dimer.

a. Starting Materials for HPLC Analysis

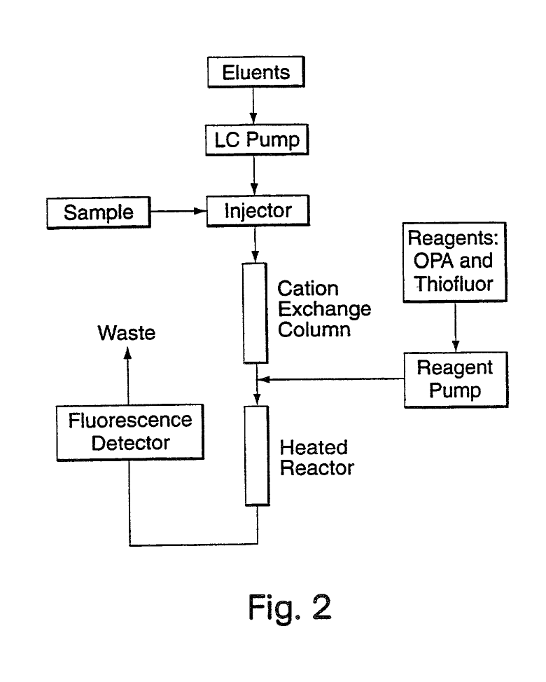

A schematic diagram of the HPLC post column reaction hardware is shown on FIG. 2.

1. Potassium phosphate eluent, Pickering laboratories Catalog No.1700- 1101; pH 6.00; 0.1 N. (Pickering Laboratories Inc., Mountain View, Calif.)

2. Potassium chloride eluent, Pickering Laboratories Catalog No. 1700-1102; pH 6.00; 0.1 N.

3. o-Phthalaldehyde (OPA) diluent, Pickering Laboratories Catalog No. OD104.

4. o-Phthalaldehyde, Pickering Laboratories Catalog No. O120.

5. Thiofluor, Pickering Laboratories Catalog No. 3700-2000.

6. Nitrogen, grade 4.8

7. Equipment and conditions

Beckman 126 solvent module (or equivalent); (Beckman Instruments Inc., Fullerton, Calif.)

Beckman Gold Nouveau software (or equivalent)

Jasco FP-920 intelligent Fluorescent detector (Jansco Inc., Easton, Md.)

Pickering Laboratories PCX 3100 post-column reaction module

Alkion cation-exchange column 4×150 mm; Pickering Laboratories

Catalog No. 9410917

Excitation Wavelength. 330 nm

Emission Wavelength: 465 nm

Column temperature: 40° C.

Reactor temperature: 45° C.

Reactor pump flow rate: 0.3 ml/min.

b. Preparation of Reagent Solutions

1. Degas 450 ml OPA diluent for approximately 10 min. by bubbling nitrogen gas through the diluent.

2. Dissolve approximately 0.05 g(OPA) in a minimum amount of methanol, add to OPA diluent.

3. Dissolve approximately 1 g thiofluor in a small amount of OPA diluent, add to OPA solution from Step 2.

c. Protocol for HPLC analysis of ethlyeneimine dimer

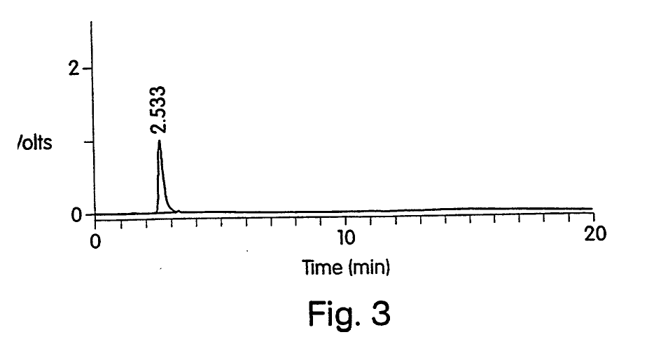

This method uses cationic exchange chromatography with post column derivatization of ethyleneimine oligomer primary amino groups and fluorescent detection. The HPLC elution profile of ethyleneimine dimer is shown on FIG. 3.

1. Prepare samples in potassium phosphate eluent, pH 6. Dilute 1 μl ethyleneimine dimer at a final concentration of 1:8×10⁴ with the eluent.

2. Use potassium chloride eluent pH 6.

3. Inject 10 μl of the sample for analysis. Flow rate is set at 0.8 ml/min.

4. 0-4 min., 100% flow potassium phosphate eluent; 4-8 min., 0-100% flow potassium chloride eluent; 9-10 min., 100-0% flow potassium chloride eluent.

5. Reequilibrate the column with 100% potassium phosphate eluent for an additional 10 min. between analyses.

Since this method is very sensitive to contamination, gloves should be worn at all times when preparing buffers or samples.

The following specific examples are to be construed merely as illustrative, and not limitative of the remainder of the disclosure in any way whatsoever. Furthermore, although some of the examples describe the addition of the ethyleneimine dimer to whole blood prior to the isolation of blood components, preferably the desired blood component (e.g., red blood cells) is isolated prior to addition of ethyleneimine dimer. This not only reduces the amount of ethyleneimine dimer required to inactivate the viruses contained in the desired blood component, but also allows the retainment of other, untreated blood components (e.g., plasma), which may be subsequently (or simultaneously) virally inactivated with ethyleneimine dimer. For example, virally inactivated plasma may be used to purify virus-free plasma proteins, such as blood clotting factors or albumin.

### EXAMPLE I

### Biochemistry of Baboon RBC Treated with Ethyleneimine Dimer or Ethyleneimine Trimer

Fifteen milliliters of fresh baboon blood was collected from baboon 205 (Naval Blood Research Laboratory) in CPD-ADSOL (a standard blood storage solution that prevents the blood from clotting). The fresh whole blood was treated with 0.79 ml of 240 mM ethyleneimine dimer in 0.25 M NaH₂PO₄ (20X stock solution of ethyleneimine dimer was prepared immediately before the treatment). The final concentration of ethyleneimine dimer in the blood was 12 mM (1000 μg/ml; 0.1% v/v). In a control experiment, 0.79 ml of 0.25 M NaH₂PO₄ was added to 15 ml of fresh baboon blood. Both control and ethyleneimine dimer-treated samples were incubated, with rocking, for six hours at room temperature. After the end of the incubation period, the red blood cells were isolated by centrifugation, and a series of biochemical parameters were immediately determined without removal of ethyleneimine dimer.

In addition, a parallel experiment was performed with a second ethyleneimine oligomer, ethyleneimine trimer. As above, 15 ml of fresh baboon blood was collected from baboon 205 (Naval Blood Research Laboratory) in CPD-ADSOL. The fresh whole blood was treated with 0.79 ml of 310 mM ethyleneimine trimer in 0.5 M NaH₂PO₄.(20X stock solution of ethyleneimine trimer was prepared immediately before the treatment). The final concentration of cthyleneimine trimer in the blood was 15.5 mM (2000 μg/ml; 0.2% v/v). In a control experiment, 0.79 ml of 0.5 M NaH₂PO₄ was added to 15 ml of fresh baboon blood. Both control and ethyleneimine trimer-treated samples were incubated, with rocking, for six hours at room temperature (i.e., 22° C.). After the end of the incubation period, the red blood cells were isolated by centrifuigation, and a series of biochemical parameters were immediately determined without removal of ethyleneimine trimer.

Table I shows the results of the RBC biochemistry tests.

TABLE I
Biochemistry of CPD-ADSOL Baboon RBC Treated
With 12 mM Ethyleneimine Dimer or 15.5 mM Ethyleneimine Trimer for 6 Hours at 22° C.

| | pH | | | | | MCV | MCHC | | Red Cell K⁺ | Red Cell Na⁺ | | ATP |
|---|---|---|---|---|---|---|---|---|---|---|---|---|
| | 22° C. | MOS | SUP HB | HB | spun | (spun | (g/dL of | SUP K⁺ | (meq/10₁₂ | (meq/10¹² | P50 | (μmole/ |
| | (WB) | mOs/kg | (mg/dL) | (g/dL) | HCT | HCT) | RBC) | (mEq/L) | RBC) | RBC) | (mmHg) | gHb) |
| control | 7.16 | 396 | 29 | 15.9 | 48 | 79 | 33 | 3.5 | 7.9 | 1.4 | 27.2 | 1.7 |
| ethyleneimine | 7.16 | 404 | 25 | 16.1 | 48 | 78 | 34 | 3.6 | 7.9 | 1.2 | 29.1 | 1.7 |
| dimer | | | | | | | | | | | | |
| ethyleneimine | 7.16 | 424 | 24 | 15.9 | 46 | 76 | 35 | 2.7 | 7.7 | 1.4 | 28.9 | 1.5 |
| trimer | | | | | | | | | | | | |

As can be seen from Table I, no significance difference was found between the control (i.e., untreated), the ethyleneimine dimer-treated, or the ethyleneimine trimer-treated blood. Hence, treatment with ethyleneimine dimer or ethyleneimine trimer does not appear to affect the biochemical functioning of red blood cells.

### EXAMPLE II

### Biochemistry and in Vivo Survival of Ethyleneimine Dimer Treated and Washed Baboon RBC

IIa. Treatment of fresh CPD baboon blood with ethyleneimine dimer

Eighty milliliters of fresh baboon blood collected from baboon 214 (Naval Blood Research Laboratory) in CPD (resulting in a spun HCT of 38%) was treated with 4.2 ml of 240 mM ethyleneimine dimer in 0.25 M NaH₂PO₄ (20X stock solution of ethyleneimine dimer was prepared immediately before treatment). The final calculated total concentration of ethyleneimine dimer in the blood was 12 mM (1000 μg/ml; 0.1% v/v).

IIb. Distribution of ethyleneimine dimer between RBC and plasma in fresh whole CPD baboon blood.

Following 6 hours of incubation of the blood at room temperature, the blood was separated into a plasma fraction and a red blood cell (RBC) fraction. The concentration of ethyleneimine dimer in the plasma and RBC fractions was determined using the HPLC analysis described above.

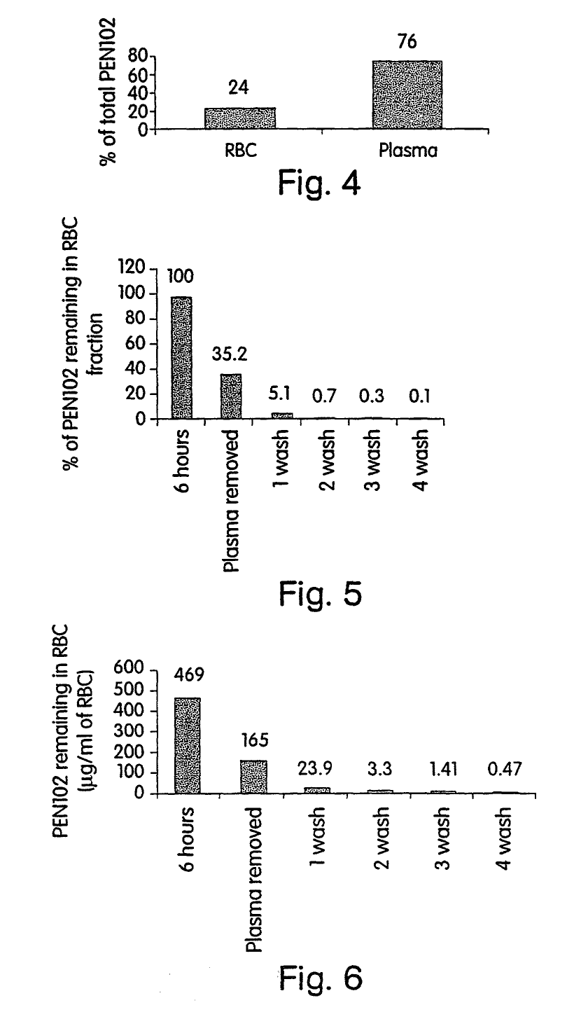

The concentration of ethyleneimine dimer in the plasma fraction was found to be 10.7 mM (893 μg/ml), while the concentration of ethyleneimine dimer in the RBC fraction was only 5.6 mM (469 μg/ml). Therefore, after six hours of incubation, the RBC and plasma fractions of blood contained 24% and 76% of the total ethyleneimine dimer respectively (FIG. 4).

IIc. Manual washing of baboon RBC after ethyleneimine dimer treatment.

Eighty milliliters of treated baboon blood was divided in six portions (about 13 ml each) in 50 ml sterile tubes. The average volume of RBC in each portion was about 5 ml. Unbuffered sterile saline (i.e., 0.9% NaCl in water) was added to fill each tube to about 50 ml. The RBC fraction was separated by centrifugation at 2000 rpm (1248×g) for 5 min. at room temperature, and the diluted plasma fraction removed.

The RBC fraction was next subjected to a washing cycle: To the remaining RBC (about 5 ml in each tube), a new portion of unbuffered sterile saline (about 4.5 ml) was added. The tubes were incubated with gentle agitation at room temperature for 10 min. At the end of the incubation, RBC fraction was separated by centrifugation. After each cycle, a small aliquot of the RBC suspension was removed to determine the concentration of ethyleneimine dimer by HPLC (see method above).

The washing cycle was repeated four times. After the fourth cycle, the six tubes of RBC were combined together and unbuffered sterile saline added to give a final hematocrit (HCT) of 46%. The effectiveness of the washing procedure is shown on FIG. 5, which demonstrates a rapid reduction in the percentage of ethyleneimine dimer in the RBC fraction. The concentration of ethyleneimine dimer remaining in the RBC fraction (in μg/ml of RBC) after each washing cycle is shown on FIG. 6. The data in FIG. 6 demonstrate that the residual concentration of ethyleneimine dimer in the RBC after four washings was lower than the safety level for ethyleneimine dimer, 1 μg/ml, which was determined by mutagenicity testing on mouse lymphoma cells (performed by Covance, Vienna, Va.).

IId. Biochemistry of baboon RBC after ethyleneimine dimer treatment, washing, and storage for 18 hours.

Washed, treated RBC (see section IIc above) were resuspended in 0.9% saline-0.2% glucose to a hematocrit (HCT) of about 46%, and stored overnight (18 hours) at 4° C. Following storage, a series of biochemical parameters were determined. The results of biochemistry tests performed on these treated, washed, and stored RBC are presented in Table II.

TABLE II
Biochemistry of Control (Untreated) and Ethyleneimine Dimer-Treated,
Washed, and Stored Baboon RBC

| | | | | | MCV | | | | | MCHC |
|---|---|---|---|---|---|---|---|---|---|---|
| | MOS | SUP HB | HB | spun | (spun | SUP K+ | Red Cell | P50 | RBC | (spun |
| | mOs/kg | (mg/dL) | (g/dL) | HCT | HCT) | (mEq/L) | K⁺ | (mmHg) | pH | HCT) |
| control* | 394 | 3 | 12.6 | 38 | 80.9 | 3.5 | 7.9 | 31.8 | 6.939 | 29.0 |
| ethyleneimine | NA | 90 | 13.3 | 46 | 91.1 | 2.2 | 8.0 | 30.6 | 6.649 | 32.0 |
| dimer** | | | | | | | | | | |

*fresh, untreated CPD baboon blood
**baboon CPD blood, treated with ethyleneimine dimer, washed, and stored for 18 hours at 4° C.

As shown in FIGS. 5 and 6, four manual washing cycles of treated RBC effectively reduced the concentration of ethyleneimine dimer below the level of safety determined in a companion mutagenicity study. Table II demonstrates that treatment of RBC with ethyleneimine dimer, followed by washing and 18 hours of storage at 4° C., did not affect the major biochemical characteristics of the RBC.

IIe. Post-treatment in vivo survival of RBC.

Five milliliter portions of ethyleneimine dimer treated RBC from baboon 214 were labeled with either ⁵¹Cr or biotin using standard labeling protocols (see, e.g., Valeri et al., Transfusion 24: 105-108, 1984). For example, to label RBCs with ⁵¹Cr, approximately 20-30 ml of RBCs were incubated for 30 min. at 37° C. with 0.5 μCi ⁵¹Cr (disodium chromate) per ml of blood (commercially available from, for example, Dupont/NEN, Boston, Mass.), and then washed to remove the unincorporated ⁵¹Cr-label. The RBCs were biotin-labeled using the Biotin-X-NHS kit, commercially available from Calbiochem (San Diego, Calif.). The ⁵¹Cr-labeled RBCs and biotin-labeled RBCs were then combined and infused back into baboon 214. Table III shows the in vivo survival time of the ethyleneimine dimer treated, washed, and stored RBC compared to the in vivo survival time of normal RBCs that had been removed from baboon 214, labeled, and re-infused. The half-life (T50) of the RBCs is shown in days.

TABLE III
In Vivo Survival of RBC Post-Ethyleneimine Dimer Treatment

| | T50 ⁵¹Cr | T50 biotin |
|---|---|---|
| Experimental | 12.2 days | 33 days |
| Historical for Baboon 214 | 13.8 days +/− 1.1 days | 38 days +/− 5 days |

As Table III demonstrates, the treatment of baboon RBC with 12 mM ethyleneimine dimer, followed by manual washing and storage of the RBC for 18 hours at 4° C., did not affect either the biochemistry or the lifespan of the RBC.

### EXAMPLE III

### Quenching Ethyleneimine Dimer in Treated Human Blood with Na-Thiosulfate or a Solid-Phase Quencher

In another method to remove ethyleneimine dimer from treated red blood cells, a quenching agent, either soluble (i.e., Na-thiosulfate) or solid-phase, was used. It should be noted that the reagents and methods described in this example are described in more detail in Purmal et al., Solid Phase Quenching Systems, (U.S. patent application Ser. No. 09/161,078, filed Sep. 25, 1998), hereby incorporated by reference.

In this experiment, 50 μl of 120 mM ethyleneimine dimer in 0.25 M NaH₂PO₄ was added to 0.9 ml of whole human CPD blood (final concentration of ethyleneimine dimer was 6 mM, 6 μmole total), and incubated at 23° C. for 4 hours.

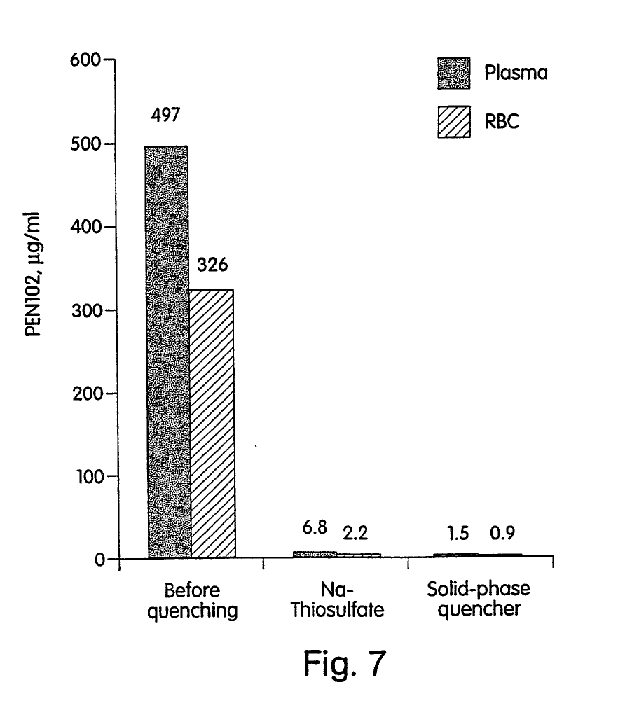

At the end of the 4-hour incubation period, 68 mg (50 μmole-equivalents of phosphothiomonoester groups) of ArgoPore-Thiophosphate support was added. In the parallel experiment, 50 μl of 1 M Na₂S₂O₃ (final concentration 50 mM) was added to same amount of ethyleneimine dimer treated blood. Both samples were allowed to incubate for two hours at 23° C. The red blood cell (RBC) and plasma fraction of the blood were separated by centrifugation (10,000 rpm, 5 min.), and the RBCs were opened by adding nine volumes of water. The concentration of ethyleneimine dimer was determined in RBC and in the plasma fraction of the blood by HPLC (FIG. 7).

As shown in FIG. 7, both sodium thiosulfate and solid phase-bound thiophosphate groups were capable of quenching ethyleneimine dimer. After 2 hours, the plasma quenched with sodium thiosulfate contained only 6.8 μg/ml ethyleneimine dimer, and the red blood cells quenched with sodium thiosulfate contained 2.2 μg/ml ethyleneimine dimer. The solid phase quencher containing thiophosphate groups was even more effective. The plasma quenched with this system contained only 1.5 μg/ml ethyleneimine dimer, and the red blood cells contained only 0.9 μg/ml ethyleneimine dimer after two hours. Thus, incubation of ethyleneimine dimer treated red blood cells with the solid-phase quencher for two hours lowered the concentration of ethyleneimine dimer in the red blood cells to a non-toxic level.

### EXAMPLE IV

### In Vivo Survival of Ethyleneimine Dimer Treated, Washed, and Frozen Canine RBC

Blood is collect from a dog and divided into two portions. The first portion is incubated with an amount of ethyleneimine dimer sufficient to inactivate at least some of the animal viruses in the portion. Following incubation, the red blood cells from this treated portion are isolated, washed four times with sterile unbuffered saline, as described above, and then frozen in glycerol for two weeks at −70° C.

At the same time, the second portion is incubated for six hours at 22° C., but without any addition of ethyleneimine dimer. Following incubation, the red blood cells are isolated and washed four times with sterile saline, and then frozen in glycerol for two weeks at −70° C.

Following the two weeks of frozen incubation time, the cells are thawed. and cells in the first portion (i.e., the ethyleneimine dimer-treated portion) are labeled with ⁵¹Cr while the cells in the second portion (i.e., the untreated portion) are labeled with biotin. The cells are then combined and re-infused to the donor dog. The survival time of the ⁵¹Cr labeled red blood cells is compared to the survival time of the biotin-labeled cells, and no significant difference is predicted to be found between the two. Hence, ethyleneimine dimer treatment does not affect the survival time of canine red blood cells in vivo.

### EXAMPLE V

### In Vivo Survival of Ethyleneimine Dimer Treated, Washed, and Frozen Baboon Platelets

Eighty milliliters of fresh baboon blood is collected from a baboon and the platelets are immediately separated from the remaining blood components (which are discarded). The purified platelets are divided into two equal portions, one of which (i.e., the ethyleneimine dimer treated portion) is treated for six hours at room temperature with 2.1 ml of 240 mM ethyleneimine dimer in 0.25 M NaH₂PO₄ (20X stock solution of ethyleneimine dimer is prepared immediately before treatment) with the final calculated total concentration of ethyleneimine dimer in the platelet-containing solution being 12 mM (1000 μg/ml; 0.1% v/v). The second portion (i.e., the untreated portion) is treated for six hours at room temperature with 2.1 ml of a 0.25 M NaH₂PO₄.

The two portions of platelets are next separated by centrifugation, and the supernatant removed. The pelleted cells are next resuspended in sterile unbuffered saline, incubated at room temperature on a rocker for 10 min., and re-pelleted by centrifugation. After four cycles of this resuspension/re-pelleting, the platelets are frozen in glycerol for two weeks at −70° C.

After two weeks, both the treated and the untreated portions of platelets are thawed and labeled with ⁵¹Cr, as described above. In a blind study, the ⁵¹Cr labeled ethyleneimine dimer treated platelets are transfused into one recipient baboon and the ⁵¹Cr labeled untreated platelets are transfused into a second recipient baboon. Both of the recipient baboons are the same gender and roughly the same age. The T50 of the ethyleneimine dimer treated platelets is predicted to be approximately equal to that of the untreated platelets.

### EXAMPLE VI

### Biochemistry and In Vivo Survival of Ethyleneimine Dimer-Treated, Washed, and Stored Human RBC

Blood donated by a human blood donor is incubated with an amount of ethyleneimine dimer sufficient to inactivate at least some of the animal viruses in the blood. Following incubation and using the MCS®+ Apheresis System (commercially available form Haemonetics Corp., Braintree, Mass.), the ethyleneimine dimer-treated blood is separated into three components: (1) the red blood cells (RBCs), (2) the platelets, and (3) the plasma. The plasma and platelets are frozen in glycerol and stored at −70° C.

The isolated RBCs are spread onto a mesh having pores with diameters that are smaller than the diameter of a human RBC, and rinsed with a continuous flow of sterile unbuffered saline until the concentration of ethyleneimine dimer in the cells is at or below the concentration determined to be non-toxic in the mouse lymphoma forward mutation assay described above. The washed cells are next stored for 18 hours at 4° C. The sample of cells is then divided into two. The first of the two portions of washed, treated, and stored RBCs are subjected to testing for a series of biochemical parameters. No significant changes in the biochemical characteristics of the washed, treated RBC are predicted to be seen as compared to a sample of washed, untreated RBC.

The second of the two portions of washed, treated, and stored RBCs are labeled with biotin, and re-infused back into the same human donor. Tracking of the biotin-labelled cells demonstrates that the ethyleneimine dimer-treated cells survive in vivo for a length of time comparable that of untreated biotin-labeled red blood cells that are similarly isolated (i.e., using the MCS®+ Apheresis System), washed, and stored.

### EXAMPLE VII

### Protein Purification from Ethyleneimine Dimer-Treated and Washed Bovine Urine

A first sample of bovine urine is collected and incubated with an amount of ethyleneimine dimer sufficient to inactivate at least some of the viruses in the urine. Contemporaneously, a second sample of bovine urine is collected and subjected to the same incubation conditions as the first sample, but in the absence of ethyleneimine dimer. Both urine samples are next greatly diluted with sterile water until the concentration of the ethyleneimine dimer is below that determined to be toxic in a mouse lymphoma mutagenicity assay (such as that described above and commercially performed by Covance, Vienna, Va.).

The urine from both samples is then subjected to protein purification techniques to isolate the desired urine protein (e.g., the Tamm-Horsfall glycoprotein). Standard protein purification techniques include HPLC, and described in general technique laboratory manuals (see, e.g., Scopes, R. K., Protein Purification: Principles and Practice, ed. C. R. Cantor, Spring-Verlag Inc., New York, N.Y., 1982; Coligan, J. E., Current Protocols in Protein Science, John Wiley & Sons, New York, N.Y., 1996). A comparable of amount of the desired urine protein is predicted to be purified from both the ethyleneimine dimer-treated urine and the untreated urine.

### Other Embodiments

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

Without further elaboration, it is believed that one skilled in the art can, based on the description herein, utilize the present invention to its fullest extent. All publications and patent applications mentioned in this specification are herein incorporated by reference to the same extent as if each independent publication or patent application was specifically and individually indicated to be incorporated by reference.

What is claimed is:

1. A method of inactivating microorganisms in a preparation of purified mammalian enucleated cells for therapeutic use, said method comprising the step of contacting said preparation with ethyleneimine dimer under conditions and for a period of time sufficient to inactivate at least some of the microorganisms in said preparation.

2. The method of claim 1, wherein said cells are red blood cells.

3. The method of claim 1, wherein said cells are platelets.

4. The method of claim 1, wherein said microorganisms are cell-contained microorganisms.

5. The method of claim 1, wherein said microorganisms comprise viruses.

6. The method of claim 5, wherein said viruses comprise at least one virus selected from the group consisting of hepatitis A virus, hepatitis B virus, hepatitis C virus, human immunodeficiency virus, and parvovirus.

7. The method of claim 1, wherein said microorganisms comprise bacteria.

8. The method of claim 1, wherein said microorganisms comprise parasites.

9. The method of claim 1, wherein at least 90% of the microorganisms in said preparation are inactivated.

10. The method of claim 9, wherein at least 98% of the microorganisms in said preparation are inactivated.

11. The method of claim 1, further comprising the step of transfusing said ethyleneimine dimer-contacted preparation into a mammal.

12. The method of claim 11, wherein at least some of said ethyleneimine dimer is removed prior to said transfusing.

13. The method of claim 12, wherein said ethyleneimine is removed by washing said ethyleneimine dimer-contacted preparation.

14. The method of claim 1 1, wherein said mammal is a human.

15. The method of claim 1, wherein said method further comprises quenching said ethyleneimine dimer with a quenching agent after said contacting step.

16. The method of claim 15, further comprising the step of transfusing said quenching agent-contacted preparation into a mammal.

17. The method of claim 16, wherein said mammal is a human.

18. The method of claim 15, wherein said quenching agent is soluble.
